(19) United States
(12) Patent Application Publication
(10) Pub. No.: US 2025/0146974 A1
(43) Pub. Date: May 8, 2025

(54) METHOD FOR DISSOLVING SINGLE-PARTICLE TITANITE AND METHOD FOR DETERMINING AGE OF SINGLE-PARTICLE TITANITE BY(URANIUM-THORIUM)/HELIUM DATING

(71) Applicant: Institute Of Geology, Chinese Academy Of Geological Sciences, Beijing (CN)

(72) Inventors: Wen CHEN, Beijing (CN); Qiuyi DU, Beijing (CN); Jingbo SUN, Beijing (CN); Yuntao TIAN, Beijing (CN); Ze SHEN, Beijing (CN); Bin ZHANG, Beijing (CN); Ziman GUO, Beijing (CN)

(21) Appl. No.: 18/926,959

(22) Filed: Oct. 25, 2024

(30) Foreign Application Priority Data
Nov. 2, 2023 (CN) 202311451668.3

(51) Int. Cl. G01N 27/626; C01G 23/00; G01N 1/38; G01N 1/44; G01N 33/00

(52) U.S. Cl. CPC G01N 27/626; C01G 23/002; G01N 1/38; G01N 1/44; G01N 33/0004

(57) ABSTRACT

Disclosed are a method for dissolving a single-particle titanite and a method for determining an age of a single-particle titanite by (uranium-thorium)/helium dating, relating to the technical field of mineral isotope chronometry. A dissolution method exclusive to the single-particle titanite is provided. In the method for determining the age of the single-particle titanite by (uranium-thorium)/helium dating, contents of uranium, thorium, and helium are obtained by measuring a same sample, which are then substituted into a (uranium-thorium)/helium age equation to directly obtain an age value.

### CROSS REFERENCE TO RELATED APPLICATION

This patent application claims the benefit and priority of Chinese Patent Application No. 2023114516683, entitled “Method for dissolving single-particle titanite and method for determining age of single-particle titanite by (uranium-thorium)/helium dating” filed on Nov. 2, 2023, the disclosure of which is incorporated by reference herein in its entirety as part of the present application.

### TECHNICAL FIELD

The present disclosure relates to the technical field of mineral isotope chronometry, and in particular to a method for dissolving a single-particle titanite and a method for determining an age of a single-particle titanite by (uranium-thorium)/helium dating.

### BACKGROUND

In recent years, thermochronometry has developed rapidly, which could not only provide the age of geological body, but also provide the information of temperature and depth while mineral and geological body are formed. Thus, thermochronometry has a great potential in the research on geological body formation and tectonic evolution. Due to the sensitivity of the (uranium-thorium)/helium system to a low temperature, (uranium-thorium)/helium dating is one of the most common dating techniques in the current low-temperature thermochronometry. The (uranium-thorium)/helium dating technique provides a very important and effective means for geological dating, research on basin and region thermal history evolution, research on paleogeomorphology restoration and evolution, sediment source tracing, analysis of the tectono-thermal history of the source area, research on a tectonic uplift and exhumation history history, and research on mineral deposit formation and uplift and exhumation processes.

Like other isotope dating methods, the (uranium-thorium)/helium dating technique is based on the principle of radioactive decay. Parent isotopes ²³⁸U, ²³⁵U, and ²³²Th undergo a decay to produce a daughter isotope ⁴He according to decay equations (I) to (III):

$${}^{238} U \to {}^{206} Pb + 8 α ( {}^{4} He ) + 6 {β}^{-} ; \qquad ( I )$$

$${}^{235} U \to {}^{207} Pb + 7 α ( {}^{4} He ) + 4 {β}^{-} ; and \qquad ( II )$$

$${}^{232} Th \to {}^{208} Pb + 6 α ( {}^{4} He ) + 4 {β}^{-} . \qquad ( III )$$

In practice, other elements, including Sm, can also undergo a decay to produce radioactive He, but only lead to a much lower amount of ⁴He than the above parent isotopes in most cases. In laboratories, only contents of residual ²³⁸U, ²³⁵U, and ²³²Th in the mineral are usually determined. However, ⁴He produced by ²³⁸U, ²³⁵U, and ²³²Th could partially or fully retain in a mineral and gradually accumulate under appropriate conditions. Therefore, a content of ⁴He in a mineral could be determined, and then according to the above radioactive decay equations and the known parameters (λ₂₃₈=1.551×10⁻¹⁰; λ₂₃₅=9.848×10⁻¹⁰; and λ₂₃₂=4.947×10⁻¹¹), a (uranium-thorium)/helium age of the mineral could be calculated. A natural U isotope ratio ²³⁵U/²³⁸U is 1/137.88. The following ⁴He age equation is obtained according to the above equations:

$${}^{4} He = 8 \times {}^{238} U \times ( {e}^{{λ}_{238} t} - 1 ) + 7 \times ( {}^{238} U / 137.88 ) ( {e}^{{λ}_{235} t} - 1 ) + 6 \times {}^{232} Th \times ( {e}^{{λ}_{232} t} - 1 ) . \qquad ( 1 )$$

The equation (1) is tenable on the prerequisite that there is no original ⁴He in a mineral to be dated. Due to a very low content of helium in the atmosphere (about 5×10⁻⁶), the inclusion of atmospheric helium could be ignored in most cases.

Because different diffusion kinetics of helium in different minerals lead to different closure temperatures, helium ages of different minerals could provide information of thermal history evolutions of different temperature ranges, and a cooling history of a geological body could be obtained with a (uranium-thorium)/helium system. A large number of minerals may be suitable for (uranium-thorium)/helium dating, but only a few minerals such as zircon and apatite have been investigated in detail. Titanite is a nesosilicate mineral of a monoclinic system. Titanite includes uranium and thorium, and is widely distributed in various rocks such as magmatic rocks and metamorphic rocks in the form of an auxiliary mineral. As a result, titanite has attracted attention as a potential mineral for (uranium-thorium)/helium dating. It is determined through research on helium diffusion characteristics inside titanite that the closure temperature of a helium system in titanite is 100° C. to 180° C., and this temperature range cannot be well constrained by other existing thermochronology techniques. The temperature range for a fission track of apatite is about 60° C. to 120° C., and an argon-argon system of feldspar minerals could provide information in a temperature range of higher than 170° C. Therefore, the application of a (uranium-thorium)/helium system of titanite could fill the gap in low-temperature thermochronometry research.

There are many research findings of (uranium-thorium)/helium dating techniques inside and outside China, but there are relatively few (uranium-thorium)/helium dating methods for titanite. Sporadic published results are to use titanite aliquots to conduct (uranium-thorium)/helium dating or completely copy a (uranium-thorium)/helium dating method for zircon to determine a (uranium-thorium)/helium age of titanite.

A specific method of using titanite aliquots to conduct (uranium-thorium)/helium dating is as follows: two titanite samples in equal amounts are prepared; and one of the two titanite samples is used to determine the helium content, and the other one of the two titanite samples is used to determine contents of uranium and thorium. A serious drawback of this method is that there is a minimum mass limit for each aliquot. Because there are inconsistent contents of uranium and thorium in sample particles, each aliquot must be large enough to equalize differences in contents of uranium and thorium among titanite particles. An amount of each aliquot could only be estimated through attentive investigation of a sample, and an estimated aliquot amount has poor accuracy. Generally, an aliquot requires at least a few milligrams of a sample, resulting in very large titanite sample consumption. (Uranium-thorium)/helium dating with aliquots is a relatively primitive method of (uranium-thorium)/helium dating, but with the improvement of experimental procedures, this method with poor accuracy and large sample consumption is gradually replaced by (uranium-thorium)/helium dating with a single-particle sample.

A single-particle (uranium-thorium)/helium dating method has been established for minerals such as zircon and apatite inside and outside China, and in this method, contents of uranium, thorium, and helium could be obtained by measuring a same sample. A dissolution procedure is a key in (uranium-thorium)/helium dating of a single-particle sample. Common dissolution methods include atmospheric pressure digestion, high-pressure digestion, ultrasonic-wave digestion, or the like. Apatite is a relatively-soluble mineral, which could be completely dissolved by adding an acid reagent to a sample and subjecting the resulting mixture to an ultrasonic treatment. However, silicate minerals such as zircon and titanite are difficult to dissolve, and the dissolution of these silicate minerals requires multiple times of digestion with a variety of acid reagents. When the atmospheric pressure digestion is adopted, an open container is generally used. As a result, the atmospheric pressure digestion not only cannot completely dissolve some insoluble elements, but also pollutes the environment and endangers the health of experimenters due to volatilization of a large amount of acids at a high temperature. In addition, the atmospheric pressure digestion is easy to cause a loss of some elements due to volatilization, which seriously affects an accuracy of an analysis result. In a (uranium-thorium)/helium dating method for zircon, a zircon sample is dissolved through high-pressure digestion with a highly-corrosive acid such as hydrofluoric acid, in which a single time of digestion is conducted at 200° C. or higher for 48 h or more. In addition, re-dissolution is required in this method, and thus the entire sample dissolution procedure costs relatively long time. If the zircon sample dissolution procedure is directly applied to titanite, the dissolution time is long, dissolution efficiency is low, and there is no guarantee that a titanite sample could be completely dissolved.

Currently, there are no reports on the successful establishment of a (uranium-thorium)/helium dating experimental method for single-particle titanite.

### SUMMARY

An object of the present disclosure is to provide a method for dissolving a single-particle titanite and a method for determining an age of a single-particle titanite by (uranium-thorium)/helium dating. The method makes it possible to accurately determine a (uranium-thorium)/helium age, which fills a gap of (uranium-thorium)/helium dating for single-particle titanite.

To achieve the above object of the present disclosure, the present disclosure provides the following technical solutions.

Provided is a method for dissolving a single-particle titanite, including the following steps:
mixing the single-particle titanite, hydrofluoric acid, and a concentrated nitric acid to obtain a first mixture, and subjecting the first mixture to thermal digestion at 180° C. for 24 h in an autoclave, to obtain a primary dissolved sample;
heating the primary dissolved sample and evaporating all liquid therein, to obtain a dry sample; and
mixing the dry sample with a concentrated hydrochloric acid to obtain a second mixture, and subjecting the second mixture to re-dissolution in an autoclave at 180° C. for 24 h to obtain a re-dissolved sample.

In some embodiments, the hydrofluoric acid is added in an amount of 350 μL; the concentrated nitric acid is added in an amount of 25 μL; and the concentrated nitric acid has a volume concentration of 50%.

In some embodiments, the concentrated hydrochloric acid is added in an amount of 300 μL.

In some embodiments, the hydrofluoric acid, the concentrated nitric acid, and the concentrated hydrochloric acid each independently contain lower than 0.01 ppb of a metal impurity.

In some embodiments, heating the primary dissolved sample and evaporating all liquid therein is conducted at 60° C.

The present disclosure provides a method for determining an age of a single-particle titanite by (uranium-thorium)/helium dating, including the following steps:
S1: selecting a single-particle titanite sample;
S2: heating the single-particle titanite sample and extracting ⁴He therefrom, and purifying a resulting gas to obtain a purified gas; and determining a content of ⁴He in the purified gas by an isotope dilution method using a helium isotope mass spectrometer (MS), namely a content of ⁴He in the single-particle titanite sample;
S3: dissolving the single-particle titanite sample according to the method as described in above solutions to obtain a mixed solution to be tested, wherein the thermal digestion is conducted as follows: mixing the single-particle titanite sample with a spike and hydrofluoric acid to obtain a first mixture, and subjecting the first mixture to the thermal digestion; and determining contents of ²³⁸U and ²³²Th in the single-particle titanite sample by an isotope dilution method using an inductively coupled plasma mass spectrometer (ICP-MS), wherein the spike is a concentrated nitric acid solution including ²³⁵U, ²³⁸U, ²³²Th, and ²³⁰Th; and
S4: substituting determined contents of ⁴He, ²³⁸U, and ²³²Th in the single-particle titanite sample into age equation (1), and calculating a (uranium-thorium)/helium age of the single-particle titanite sample,

$$equation ( 1 )$$

$${}^{4} He = 8 \times {}^{238} U \times ( {e}^{{λ}_{238} t} - 1 ) + 7 \times ( {}^{238} U / 137.88 ) ( {e}^{{λ}_{235} t} - 1 ) + 6 \times {}^{232} Th \times ( {e}^{{λ}_{232} t} - 1 ) ,$$

wherein in equation (1), 4He, ²³⁸U, and ²³²Th each represent a measured number of atoms; t represents an accumulated time of a radioactive decay for producing a daughter isotope ⁴He; and λ₂₃₈, λ₂₃₅, and λ₂₃₂ represent decay constants of ²³⁸U, ²³⁵U, and ²³²Th, respectively, which are 1.55125×10⁻¹⁰ a⁻¹, 9.8485×10⁻¹⁰ a⁻¹, and 4.9475×10⁻¹¹ a⁻¹, respectively.

In some embodiments, the single-particle titanite sample has a minimum width of larger than 80 μm.

In some embodiments, heating the single-particle titanite sample and extracting ⁴He therefrom is conducted in a 970 nm diode laser with a laser current of 15 A for 10 min.

In some embodiments, in S2, determining the content of 4He in the purified gas includes the following steps:
mixing the purified gas with a spike ³He to obtain a sample mixed gas, and determining a ⁴He/³He ratio in the sample mixed gas using a helium isotope MS, which is denoted as (⁴He/³He) spiked Sample;
mixing a ⁴He standard gas in a known amount with the spike ³He to obtain a standard mixed gas, and determining a ⁴He/³He ratio in the standard mixed gas using the helium isotope MS, which is denoted as (⁴He/³He) Spike Q standard, wherein a volume of the spike ³He used for preparation of the sample mixed gas is the same as a volume of the spike ³He used for preparation of the standard mixed gas; and calculating a content of 4He in the purified gas according to equation (2):

$$equation ( 2 )$$

$${}_{}^{4} {He}_{Sample}^{} = {}_{}^{4} {He}_{Q Standard}^{} \times [ {( {}^{4} He / {}^{3} He )}_{Spiked Sample} / {( {}^{4} He / {}^{3} He )}_{Spiked Q Standard} ] ,$$

wherein in equation (2), ⁴He_{Sample} represents a content of ⁴He in the purified gas; and ⁴He_{Q} Standard represents a content of ⁴He in the ⁴He standard gas.

In some embodiments, S3 includes:
providing the concentrated nitric acid solution including ²³⁵U, ²³⁸U, ²³²Th, and ²³⁰Th as the spike, wherein a ²³⁵U/²³⁸U ratio and a ²³⁰Th/²³²Th ratio in the spike are calibrated;
providing a nitric acid solution with known ²³⁸U and ²³²Th contents and no ²³⁰Th as a standard solution, wherein a ²³⁵U/²³⁸U ratio in the standard solution is calibrated;
mixing the single-particle titanite sample with the spike and the hydrofluoric acid to obtain the first mixture, and subjecting the first mixture to the thermal digestion, the heating and evaporating, and the re-dissolution with the concentrated hydrochloric acid sequentially to obtain the mixed solution to be tested;
mixing the standard solution with the spike and the hydrofluoric acid to obtain a third mixture, and subjecting the third mixture to the thermal digestion, the heating and evaporating, and the re-dissolution with the concentrated hydrochloric acid sequentially to obtain a spike/standard solution mixture, wherein a volume of the spike used for preparation of the mixed solution to be tested is the same as a volume of the spike used for preparation of the spike/standard solution mixture;
determining a ²³⁵U/²³⁸U ratio and a ²³⁰Th/²³²Th ratio in the mixed solution to be tested and the spike/standard solution mixture using the ICP-MS;
according to equation (3), calculating a content of ²³⁸U in the spike, which is denoted as ²³⁸U_{Spike}; and then according to equation (4), calculating a content of ²³⁸U in the single-particle titanite sample, which is denoted as ²³⁸U_{Sample}:

$${}_{}^{238} {U}_{Spike}^{} = {}_{}^{238} {U}_{Standard}^{} \times \frac{{( \frac{{}^{235} U}{{}^{238} U} )}_{Standard} - {( \frac{{}^{235} U}{{}^{238} U} )}_{mix}}{{( \frac{{}^{235} U}{{}^{238} U} )}_{mix} - {( \frac{{}^{235} U}{{}^{238} U} )}_{Spike}} , \qquad equation ( 3 )$$

wherein in equation (3), ²³⁸U_{Standard} represents a definite number of ²³⁸U atoms added from the standard solution to the spike/standard solution mixture; (²³⁵U/²³⁸U) Standard represents calibrated ²³⁵U/²³⁸U ratio in the standard solution; (²³⁵U/²³⁸U) Spike represents calibrated ²³⁵U/²³⁸U ratio in the spike; and (²³⁵U/²³⁸U) mix represents a ²³⁵U/²³⁸U ratio in the spike/standard solution mixture determined by the ICP-MS; and

$${}_{}^{238} {U}_{Sample}^{} = {}_{}^{238} {U}_{Spike}^{} \times \frac{{( \frac{{}^{235} U}{{}^{238} U} )}_{spike - sample} - {( \frac{{}^{235} U}{{}^{238} U} )}_{Spike}}{{( \frac{{}^{235} U}{{}^{238} U} )}_{Sample} - {( \frac{{}^{235} U}{{}^{238} U} )}_{spike - sample}} , \qquad equation ( 4 )$$

wherein in equation (4), ²³⁸U_{Spike} represents a definite number of ²³⁸U atoms added from the spike to the spike/standard solution mixture, which is calculated according to equation (3), and equivalent to a definite number of ²³⁸U atoms added from the spike to the mixed solution to be tested; (²³⁵U/²³⁸U) Spike represents calibrated ²³⁵U/²³⁸U ratio in the spike; (²³⁵U/²³⁸U) Sample represents a natural ²³⁵U/²³⁸U ratio in the single-particle titanite sample; and (²³⁵U/²³⁸U) spike-sample represents a ²³⁵U/²³⁸U ratio in the mixed solution to be tested determined by the ICP-MS; and according to equation (5), calculating a content of ²³²Th in the spike, which is denoted as ²³²Th_{Spike}; and then according to equation (6), calculating a content of ²³²Th in the single-particle titanite sample, which is denoted as ²³²Th_{Sample}:

$${}_{}^{232} {Th}_{Spike}^{} = {}_{}^{232} {Th}_{Standard}^{} \times \frac{{( \frac{{}^{230} Th}{{}^{232} Th} )}_{mix}}{{( \frac{{}^{230} Th}{{}^{232} Th} )}_{Spike} - {( \frac{{}^{230} Th}{{}^{232} Th} )}_{mix}} , \qquad equation ( 5 )$$

wherein in equation (5), a ²³⁰Th/²³²Th ratio in the standard solution is 0; ²³²Th_{Standard} represents a definite number of ²³²Th atoms added from the standard solution to the spike/standard solution mixture; (²³⁰Th/²³²Th) Spike represents calibrated ²³⁰Th/²³²Th ratio in the spike; and (²³⁰Th/²³²Th) mix represents a ²³⁰Th/²³²Th ratio in the spike/standard solution mixture determined by the ICP-MS; and

$${}_{}^{232} {Th}_{Sample}^{} = {}_{}^{232} {Th}_{Spike}^{} \times \frac{{( \frac{{}^{230} Th}{{}^{232} Th} )}_{Spike} - {( \frac{{}^{230} Th}{{}^{232} Th} )}_{spike - sample}}{{( \frac{{}^{230} Th}{{}^{232} Th} )}_{spike - sample}} , \qquad equation ( 6 )$$

wherein in equation (6), a ²³⁰Th/²³²Th ratio in the single-particle titanite sample is 0; ²³²Th_{Spike} represents a definite number of ²³²Th atoms added from the spike to the spike/standard solution mixture, which is calculated according to equation (5), and equivalent to a definite number of ²³²Th atoms added from the spike to the mixed solution to be tested; (²³⁰Th/²³²Th) spike represents calibrated ²³⁰Th/²³²Th ratio in the spike; and (²³⁰Th/²³²Th) spike-sample represents a ²³⁰Th/²³²Th ratio in the mixed solution to be tested determined by the ICP-MS.

In the present disclosure, a complete dissolution process for titanite particles is established. One of the major steps in the method for determining (uranium-thorium)/helium age lies in how to dissolve a mineral particle. The previous method for determining (uranium-thorium)/helium age of titanite basically copies an experimental process for zircon, where a dissolution procedure of a sample requires a high temperature, and is complicated, time-consuming, and inefficient, and involves risky experimental processes. The present disclosure provides a dissolution method exclusive to single-particle titanite. Given that titanite is a silicate mineral and hydrofluoric acid could effectively dissolve titanite, hydrofluoric acid is adopted. However, because the use of hydrofluoric acid may cause the generation of insoluble fluorides, the heating and evaporating is adopted after a sample is dissolved with hydrofluoric acid, and then the resulting product is re-dissolved with concentrated hydrochloric acid.

A titanite sample is decomposed with an acid in a closed container in the present disclosure, and thus the pressure in the closed container is increased to increase the boiling point of the acid, thereby enhancing the decomposition ability of the acid. In addition, with the closed container, volatile components could be quantitatively retained in a solution, and the dissolution temperature is relatively low. The dissolution method for single-particle titanite established in the present disclosure is performed in an autoclave, and the decomposition effect of an acid is excellent because the pressure in the autoclave is high. Compared with the zircon dissolution procedure, the method according to the present disclosure makes it possible to reduce the dissolution temperature, greatly shorten the dissolution time, thereby improving dissolution efficiency.

Further, in the present disclosure, a purified high-purity acid is adopted, and thus the interference introduced by decomposing agent could be ignored.

The present disclosure provides a method for determining a (uranium-thorium)/helium age of a single-particle titanite. In the primitive method for determining a (uranium-thorium)/helium age, contents of uranium, thorium, and helium are determined with aliquots. In the method according to the present disclosure, contents of uranium, thorium, and helium could be obtained by a test of one same sample, and then substituted into a (uranium-thorium)/helium age equation to directly obtain an age value, which avoids investigation and weighing during preparation of a sample, makes a test convenient, and also effectively saves a precious titanite sample.

In the method for determining (uranium-thorium)/helium age of a single-particle titanite according to the present disclosure, an isotope dilution method is adopted for determining contents of either a daughter isotope helium or parent isotopes uranium and thorium in titanite. The isotope dilution method has the following advantages: contents of isotopes to be measured (uranium, thorium, and helium) in a titanite sample could be determined by simply adding a spike to a standard gas/standard solution and a titanite sample at equal amounts with no need to know an exact amount of the spike. The isotope dilution method is relatively accurate, and usually has a measurement error of about 1% to 2% for uranium and thorium, and a measurement error of less than 1% for helium. Through error propagation, an age error of single-particle titanite is calculated to be less than 3%.

### BRIEF DESCRIPTION OF THE DRAWINGS

FIG. 1 shows a flow chart of a method for determining a (uranium-thorium)/helium age of a single-particle titanite according to an embodiment of the present disclosure, in which, S1 represents sample preparation, S2 represents helium content analysis, S3 represents sample dissolution, and S4 represents uranium-thorium content analysis.

FIG. 2A and FIG. 2B show measurement diagrams of a titanite particle.

FIG. 3 shows a relationship between an age of Fish Canyon Tuff (FCT) titanite and U/Th in Comparative Examples 1 to 4.

FIG. 4 shows a relationship between an age of FCT titanite and U/Th in Comparative Examples 5 and 6.

FIG. 5 shows a relationship between an age of FCT titanite and U/Th in Comparative Examples 7 and 8.

### DETAILED DESCRIPTION OF THE EMBODIMENTS

The present disclosure provides a method for dissolving a single-particle titanite, including the following steps:

mixing the single-particle titanite, hydrofluoric acid, and a concentrated nitric acid to obtain a first mixture, and subjecting the first mixture to thermal digestion at 180° C. for 24 h in an autoclave, to obtain a primary dissolved sample;

heating the primary dissolved sample and evaporating all liquid therein, to obtain a dry sample; and

mixing the dry sample with a concentrated hydrochloric acid to obtain a second mixture, and subjecting the second mixture to re-dissolution in an autoclave at 180° C. for 24 h to obtain a re-dissolved sample.

Unless otherwise specified, all the raw materials used in the present disclosure are commercially-available products well known in the art. In some embodiments of the present disclosure, the hydrofluoric acid is commercially-available pure hydrofluoric acid. In some embodiments, the concentrated hydrochloric acid is commercially-available concentrated hydrochloric acid well known in the art, and has a mass fraction of 36% to 38%.

In the present disclosure, the single-particle titanite, hydrofluoric acid, and the concentrated nitric acid are mixed to obtain a first mixture, and the first mixture is subjected to thermal digestion in an autoclave to obtain a primary dissolved sample.

In some embodiments of the present disclosure, the single-particle titanite has a minimum width of larger than 80 μm.

In some embodiments of the present disclosure, the hydrofluoric acid and the concentrated nitric acid both are purified acids, and each have a metal impurity content of lower than 0.01 ppb. In some embodiments of the present disclosure, purified high-purity acids are adopted, which could avoid the introduction of new interference by decomposition of a sample. In some embodiments of the present disclosure, the concentrated nitric acid has a volume concentration of 50% (that is to say, a volume ratio of HNO₃ to water is 1:1). In some embodiments of the present disclosure, the hydrofluoric acid is added in an amount of 350 μL. In some embodiments, the concentrated nitric acid is added in an amount of 25 μL. In the present disclosure, the control of amounts of the hydrofluoric acid and the concentrated nitric acid in the above respective ranges could not only guarantee the complete dissolution of single-particle titanite, but also guarantee the experimental safety.

In some embodiments of the present disclosure, the single-particle titanite, the hydrofluoric acid, and the concentrated nitric acid are added to a polytetrafluoroethylene (PTFE) sample-dissolving bottle, then the PTFE sample-dissolving bottle is placed in a liner of the autoclave, and hydrofluoric acid and the concentrated nitric acid with a volume concentration of 50% are then added to the liner of the autoclave; and the autoclave is sealed, and the thermal digestion is conducted therein. In some embodiments of the present disclosure, the liner of the autoclave is a PTFE liner. In some embodiments, in the liner of the autoclave, 9 mL of the hydrofluoric acid is added. In some embodiments, 420 μL of the concentrated nitric acid is added. There is a high-temperature and high-pressure environment inside the autoclave. In the present disclosure, the concentrated nitric acid and the hydrofluoric acid are added to the liner of the autoclave to maintain an internal pressure balance.

In the present disclosure, the thermal digestion is conducted at 180° C. for 24 h. In the present disclosure, when the titanite is dissolved with the hydrofluoric acid and the concentrated nitric acid, an excellent solubility is allowed, but insoluble fluorides would be produced during digestion.

In the present disclosure, after the primary dissolved sample is obtained, the primary dissolved sample is heated to evaporate all liquid therein to obtain a dry sample. In some embodiments of the present disclosure, the heating and evaporating all liquid is conducted at 60° C. In some embodiments, the heating and evaporating all liquid is conducted on a heating plate.

In the present disclosure, after the dry sample is obtained, the dry sample is mixed with concentrated hydrochloric acid to obtain a second mixture, and the second mixture is subjected to re-dissolution in an autoclave to obtain a re-dissolved sample.

In some embodiments, the concentrated hydrochloric acid is purified concentrated hydrochloric acid, and has a metal impurity content of lower than 0.01 ppb. In some embodiments of the present disclosure, the concentrated hydrochloric acid is added in an amount of 300 μL. In the present disclosure, when the concentrated hydrochloric acid is adopted for re-dissolution, the insoluble fluorides could be well dissolved.

In some embodiments of the present disclosure, the concentrated hydrochloric acid is added to the dry sample to obtain the second mixture, the second mixture is placed in a liner of the autoclave, and concentrated hydrochloric acid is then added to the liner of the autoclave; and the autoclave is sealed, and the re-dissolution is conducted. In some embodiments of the present disclosure, 9 mL of the concentrated hydrochloric acid is added to the liner of the autoclave. There is a high-temperature and high-pressure environment inside the autoclave. In the present disclosure, the concentrated hydrochloric acid is added to the liner of the autoclave to maintain an internal pressure balance.

In the present disclosure, the re-dissolution is conducted at 180° C. for 24 h.

In some embodiments of the present disclosure, the method further includes after obtaining the re-dissolved sample, removing an excess acid from the re-dissolved sample. In some embodiments of the present disclosure, removing an excess acid from the re-dissolved sample is conducted at 80° C. In the present disclosure, there is no special requirement for an implementation process of the removal of an excess acid, and any implementation processes for removing the excess acid well known in the art may be adopted. In some embodiments of the present disclosure, the re-dissolved sample is transferred by a pipette to a 7 mL PTFE sample-dissolving bottle, the 7 mL PTFE sample-dissolving bottle is heated on a heating plate, and when a volume of the re-dissolved sample in the sample-dissolving bottle is reduced to 100 μL, the heating is stopped.

The present disclosure provides a dissolution method exclusive to single-particle titanite. Given that titanite is a silicate mineral and hydrofluoric acid could effectively dissolve titanite, hydrofluoric acid is therefore adopted. However, because the use of hydrofluoric acid may cause the generation of insoluble fluorides, the heating and evaporating all liquid is adopted after a sample is dissolved with hydrofluoric acid, and a resulting product is then re-dissolved with concentrated hydrochloric acid. Compared with a zircon dissolution procedure, the method according to the present disclosure allows for reduced dissolution temperature and greatly shortened dissolution time, thereby improving dissolution efficiency.

The present disclosure provides a method for determining an age of a single-particle titanite by (uranium-thorium)/helium dating, including the steps of
S1: selecting a single-particle titanite sample;
S2: heating the single-particle titanite sample and extracting ⁴He therefrom, and purifying a resulting gas to obtain a purified gas; and determining a content of ⁴He in the purified gas by an isotope dilution method using a helium isotope mass spectrometer (MS), namely a content of ⁴He in the single-particle titanite sample;
S3: dissolving the single-particle titanite sample according to the method as described in above technical solutions to obtain a mixed solution to be tested, wherein the thermal digestion is conducted as follows: mixing the single-particle titanite sample with a spike and hydrofluoric acid to obtain a first mixture, and subjecting the first mixture to the thermal digestion; and determining contents of ²³⁸U and ²³²Th in the single-particle titanite sample by an isotope dilution method using an inductively coupled plasma mass spectrometer (ICP-MS), wherein the spike is a concentrated nitric acid solution comprising ²³⁵U, ²³⁸U, ²³²Th, and ²³⁰Th; and
S4: substituting determined contents of ⁴He, ²³⁸U, and ²³²Th in the single-particle titanite sample into age equation (1), and calculating a (uranium-thorium)/helium age of the single-particle titanite sample,

$$equation ( 1 )$$

$${}^{4} He = 8 \times {}^{238} U \times ( {e}^{{λ}_{238} t} - 1 ) + 7 \times ( {}^{238} U / 137.88 ) ( {e}^{{λ}_{235} t} - 1 ) + 6 \times {}^{232} Th \times ( {e}^{{λ}_{232} t} - 1 ) ,$$

wherein in equation (1), ⁴He, ²³⁸U, and ²³²Th each represent a measured number of atoms; t represents an accumulated time of a radioactive decay for producing a daughter isotope ⁴He; and λ₂₃₈, λ₂₃₅, and λ₂₃₂ represent decay constants of ²³⁸U, ²³⁵U, and ²³²Th, respectively, which are 1.55125×10⁻¹⁰ a⁻¹, 9.8485×10⁻¹⁰ a⁻¹, and 4.9475×10⁻¹¹ a⁻¹, respectively.

In the present disclosure, a single-particle titanite sample is first selected.

In some embodiments of the present disclosure, a clean titanite particle with a complete crystal form and without cracks and inclusions is selected as the single-particle titanite sample. Partial crystal deletion and severe crystal damage will make a measured age higher than the actual age in most cases. In addition, inclusions in titanite may cause a zoning effect of components inside a titanite crystal. Therefore, the selection of a pure titanite particle without inclusions is preferably selected as a test sample for (uranium-thorium)/helium dating. In some embodiments of the present disclosure, the single-particle titanite sample has a minimum width of larger than 80 μm, and preferably 90 μm to 120 μm. In the present disclosure, the selection of the single-particle titanite sample of the above size is conducive to test accuracy. If the size of the mineral particle is too small, an accuracy of a final age result will be seriously reduced due to a too-large correction coefficient. In some embodiments of the present disclosure, a microscope is used for observation, size measurement, and selection of the single-particle titanite sample.

In the present disclosure, after the single-particle titanite sample is selected, the single-particle titanite sample is heated to extract ⁴He, and a resulting gas is purified to obtain a purified gas; and a content of ⁴He in the purified gas is determined by an isotope dilution method using helium isotope MS, namely, a content of ⁴He in the single-particle titanite sample.

In some embodiments of the present disclosure, the single-particle titanite sample is placed into a niobium tube and then heated to extract ⁴He. In the present disclosure, there is no special requirement for a size of the niobium tube, and a niobium tube size well known in the art may be adopted. In embodiments of the present disclosure, the length and the diameter of the niobium tube both are about 1 mm. In the present disclosure, the single-particle titanite sample is placed into the niobium tube to avoid a particle loss during sample transfer and evaporation of uranium and thorium during helium content analysis. In the present disclosure, the niobium tube would not melt during helium extraction, and would also not be dissolved during subsequent dissolution to cause interference to analysis of uranium and thorium contents.

In some embodiments of the present disclosure, the heating the single-particle titanite sample and extracting ⁴He therefrom is conducted in a 970 nm diode laser. In some embodiments, a laser current of the diode laser is 15 A. In some embodiments, heating the single-particle titanite sample and extracting ⁴He therefrom is conducted for 10 min. In some embodiments, heating the single-particle titanite sample and extracting ⁴He therefrom is conducted in a 970 nm diode laser at a vacuum degree of lower than 1.0×10−8 Torr. In the present disclosure, the control of conditions of the heating and extracting ⁴He in the above respective ranges is conducive to full extraction of ⁴He.

In embodiments of the present disclosure, the single-particle titanite sample encapsuled in a niobium tube is placed in a laser chamber of the helium isotope MS, and because the loading of the sample makes the laser chamber exposed to an atmosphere, the laser chamber needs to be restored to a vacuum state and then the heating and extracting ⁴He are started. The laser chamber is vacuumized by a mechanical pump for 3 min and then by a turbo-molecular pump for 18 h until a vacuum degree in the laser chamber is lower than 1.0×10−8 Torr meeting an experimental need, and the heating and extracting ⁴He are then started.

In some embodiments of the present disclosure, in order to exclude a systematic error and an error caused by the niobium tube, and ensure an accuracy of a test, cold blank and hot blank tests are performed before the heating and extracting ⁴He.

The cold blank test refers to measuring a content of ⁴He in an instrument tube when the single-particle titanite sample encapsuled in the niobium tube is placed into the laser chamber and is not heated by a laser under the same experimental procedure; and the hot blank test refers to measuring a content of ⁴He in the instrument tube when an empty niobium tube is heated by a laser (laser current: 15 A, and heating time: 10 min) under the same experimental procedure.

When test results of the cold blank and hot blank tests are lower than or equal to 0.0040 ncc, it means that there is no impact on a measurement of ⁴He in the single-particle titanite sample. If the test result of the cold blank test is higher than the above value, it is necessary to check a vacuum degree of the instrument, whether the tube is intact, whether there is gas leakage, or the like. If the test result of the hot blank test is too high, it is necessary to check whether the empty niobium tube is contaminated.

In some embodiments of the present disclosure, the heating the single-particle titanite sample and extracting ⁴He therefrom are conducted at least twice. Under the condition that an amount of the ⁴He gas extracted at the second time is smaller than 1% of an amount of the ⁴He gas extracted at the first time or smaller than or equal to the test result of the hot blank test, the ⁴He gas extraction is considered to be sufficient, otherwise the gas extraction is continued until an amount of the ⁴He gas extracted at the last time is smaller than 1% of an amount of the ⁴He gas extracted at the first time or smaller than or equal to the test result of the hot blank test.

In some embodiments of the present disclosure, the purification is conducted by a zirconium-aluminum pump. In some embodiments, the purification is conducted for 60-120 s. In the present disclosure, the purification could effectively remove the active gases such as H₂, O₂, H₂O, CO₂, and SO₂. The purified gas is fed into the helium isotope MS for testing. In embodiments of the present disclosure, a quadrupole mass spectrometer (QMS) is adopted for analysis and testing.

In some embodiments of the present disclosure, the determining the content of ⁴He in the purified gas includes the following steps:
mixing the purified gas with a spike ³He to obtain a sample mixed gas, and determining a ⁴He/³He ratio in the sample mixed gas using a helium isotope MS, which is denoted as (⁴He/³He) Spiked Sample;
mixing a ⁴He standard gas in a known amount with the spike ³He to obtain a standard mixed gas, and determining a ⁴He/³He ratio in the standard mixed gas using the helium isotope MS, which is denoted as (⁴He/³He) spike Q standard, wherein a volume of the spike ³He used for preparation of the sample mixed gas is the same as a volume of the spike ³He used for preparation of the standard mixed gas; and
calculating a content of ⁴He in the purified gas according to equation (2):

$$equation ( 2 )$$

$${}_{}^{4} {He}_{Sample}^{} = {}_{}^{4} {He}_{Q Standard}^{} \times [ {( {}_{}^{4} He / {}_{}^{3} He )}_{Spiked Sample} / {( {}_{}^{4} He / {}_{}^{3} He )}_{Spike Q Standard} ] ,$$

wherein in equation (2), ⁴He_{Sample} represents a content of ⁴He in the purified gas; and ⁴He_{Q} Standard represents a content of ⁴He in the ⁴He standard gas.

In the present disclosure, a calculated content of ⁴He in the purified gas, namely, a content of ⁴He in the single-particle titanite sample, is expressed in a volume, and the volume needs to be converted into a molar number by an ideal-gas equation PV=nRT to calculate an age of titanite.

In the present disclosure, after the content of ⁴He in the single-particle titanite sample is determined, the single-particle titanite sample which is encapsuled in the niobium tube and subjected to ⁴He content determination in the previous step is directly dissolved according to the method described in above technical solutions to obtain a mixed solution to be tested, wherein the thermal digestion is conducted as follows: the single-particle titanite sample is mixed with a spike and the hydrofluoric acid to obtain the first mixture, and the first mixture is subjected to the thermal digestion; and contents of ²³⁸U and ²³²Th in the single-particle titanite sample are determined by an isotope dilution method using ICP-MS, wherein the spike is a concentrated nitric acid solution including ²³⁵U and ²³⁰Th.

In some embodiments, S3 includes the following specific steps:
providing the concentrated nitric acid solution comprising ²³⁵U, ²³⁸U, ²³²Th, and ²³⁰Th as the spike (commercially available), wherein a ²³⁵U/²³⁸U ratio and a ²³⁰Th/²³²Th ratio in the spike are calibrated;
providing a nitric acid solution with known ²³⁸U and ²³²Th contents and no ²³⁰Th as a standard solution (commercially available), wherein a ²³⁵U/²³⁸U ratio in the standard solution is calibrated;
mixing the single-particle titanite sample with the spike and the hydrofluoric acid to obtain the first mixture, and subjecting the first mixture to the thermal digestion, the heating and evaporating, and the re-dissolution with the concentrated hydrochloric acid sequentially to obtain the mixed solution to be tested;
mixing the standard solution with the spike and the hydrofluoric acid to obtain a third mixture, and subjecting the third mixture to the thermal digestion, the heating and evaporating, and the re-dissolution with the concentrated hydrochloric acid sequentially to obtain a spike/standard solution mixture, wherein a volume of the spike used for preparation of the mixed solution to be tested is the same as a volume of the spike used for preparation of the spike/standard solution mixture;
determining a ²³⁵U/²³⁸U ratio and a ²³⁰Th/²³²Th ratio in the mixed solution to be tested and the spike/standard solution mixture using the ICP-MS;
according to equation (3), calculating a content of ²³⁸U in the spike, which is denoted as ²³⁸U_{Spike}; and then according to equation (4), calculating a content of ²³⁸U in the single-particle titanite sample, which is denoted as ²³⁸U_{Sample}:

$${}_{}^{238} {U}_{Spike}^{} = {}_{}^{238} {U}_{Standard}^{} \times \frac{{( \frac{{}^{235} U}{{}^{238} U} )}_{Standard} - {( \frac{{}^{235} U}{{}^{238} U} )}_{mix}}{{( \frac{{}^{235} U}{{}^{238} U} )}_{mix} - {( \frac{{}^{235} U}{{}^{238} U} )}_{Spike}} , \qquad equation ( 3 )$$

wherein in equation (3), ²³⁸U_{Standard} represents a definite number of ²³⁸U atoms added from the standard solution to the spike/standard solution mixture; (²³⁵U/²³⁸U) Standard represents calibrated ²³⁵U/²³⁸U ratio in the standard solution; (²³⁵U/²³⁸U) spike represents calibrated ²³⁵U/²³⁸U ratio in the spike; and (²³⁵U/²³⁸U) mix represents a ²³⁵U/²³⁸U ratio in the spike/standard solution mixture determined by the ICP-MS; and

$${}_{}^{238} {U}_{Spike}^{} = {}_{}^{238} {U}_{Standard}^{} \times \frac{{( \frac{{}^{235} U}{{}^{238} U} )}_{spike - sample} - {( \frac{{}^{235} U}{{}^{238} U} )}_{Spike}}{{( \frac{{}^{235} U}{{}^{238} U} )}_{Sample} - {( \frac{{}^{235} U}{{}^{238} U} )}_{spike - sample}} , \qquad equation ( 4 )$$

wherein in equation (4), ²³⁸U_{Spike} represents a definite number of ²³⁸U atoms added from the spike to the spike/standard solution mixture, which is calculated according to equation (3), and equivalent to a definite number of ²³⁸U atoms added from the spike to the mixed solution to be tested; (²³⁵U/²³⁸U) Spike represents calibrated ²³⁵U/²³⁸U ratio in the spike; (²³⁵U/²³⁸U) Sample represents a natural ²³⁵U/²³⁸U ratio in the single-particle titanite sample; and (²³⁵U/²³⁸U) spike-sample represents a ²³⁵U/²³⁸U ratio in the mixed solution to be tested determined by the ICP-MS; and
according to equation (5), calculating a content of ²³²Th in the spike, which is denoted as ²³²Th_{Spike}; and then according to equation (6), calculating a content of ²³²Th in the single-particle titanite sample, which is denoted as ²³²Th_{Sample}:

$${}_{}^{232} {Th}_{Spike}^{} = {}_{}^{232} {Th}_{Standard} \times \frac{{( \frac{{}^{230} Th}{{}^{232} Th} )}_{mix}}{{( \frac{{}^{230} Th}{{}^{232} Th} )}_{Spike} - {( \frac{{}^{230} Th}{{}^{232} Th} )}_{mix}} , \qquad equation ( 5 )$$

wherein in equation (5), a ²³⁰Th/²³²Th ratio in the standard solution is 0; 232Th_{Standard} represents a definite number of ²³²Th atoms added from the standard solution to the spike/standard solution mixture; (²³⁰Th/²³²Th) spike represents calibrated ²³⁰Th/²³²Th ratio in the spike; and (²³⁰Th/²³²Th) mix represents a ²³⁰Th/²³²Th ratio in the spike/standard solution mixture determined by the ICP-MS; and

$${}_{}^{232} {Th}_{Sample}^{} = {}_{}^{232} {Th}_{Spike} \times \frac{{( \frac{{}^{230} Th}{{}^{232} Th} )}_{Spike} - {( \frac{{}^{230} Th}{{}^{232} Th} )}_{spike - sample}}{{( \frac{{}^{230} Th}{{}^{232} Th} )}_{spike - sample}} , \qquad equation ( 6 )$$

wherein in equation (6), a ²³⁰Th/²³²Th ratio in the single-particle titanite sample is 0 (natural titanite does not include ²³⁰Th, and thus ²³⁰Th/²³²Th ratios in all titanite samples are 0); ²³²Th_{Spike} represents a definite number of ²³²Th atoms added from the spike to the spike/standard solution mixture, which is calculated according to equation (5), and equivalent to a definite number of ²³²Th atoms added from the spike to the mixed solution to be tested; (²³⁰Th/²³²Th) Spike represents calibrated ²³⁰Th/²³²Th ratio in the spike; and (²³⁰Th/²³²Th) spike-sample represents a ²³⁰Th/²³²Th ratio in the mixed solution to be tested determined by the ICP-MS.

In some embodiments of the present disclosure, in order to provide a blank for a test process and monitor whether various experimental instruments and supplies such as reagents, niobium tubes, and containers used in the whole test process are contaminated, the reagents and an empty niobium tube are analyzed and tested as blanks. All reagents used in the dissolution process are added during each blank test, but a spike (namely, a substance including ²³⁵U and ²³⁰Th) is not added to a blank. Although contents of uranium and thorium in a blank could not be quantified, a blank level could be assessed based on signal levels (cps) of isotopes uranium and thorium measured by ICP-MS. In some embodiments of the present disclosure, when the contents of uranium and thorium in the single-particle titanite sample are calculated, signal levels of isotopes uranium and thorium in a blank are first deducted from signal levels of corresponding isotopes in a measured titanite sample solution. Experimental results show that, a signal level of an isotope in the single-particle titanite sample after the spike is added and the single-particle titanite sample is dissolved is about 105 times or more a signal level of a corresponding isotope in a blank, which has almost no impact on an age result of the single-particle titanite sample.

The method for dissolving a single-particle titanite and method for determining age of a single-particle titanite by (uranium-thorium)/helium dating according to the present disclosure are described in detail below with reference to examples, but these examples shall not be understood as a limitation to the scope of the present disclosure.

### Example 1

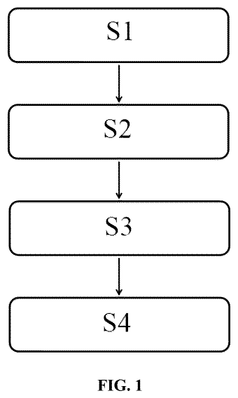

According to the flowchart shown in FIG. 1, sample preparation (S1), helium content analysis (S2), sample dissolution (S3), and uranium-thorium content analysis (S4) were conducted sequentially, and specific steps were as follows:

### (1) Sample Preparation

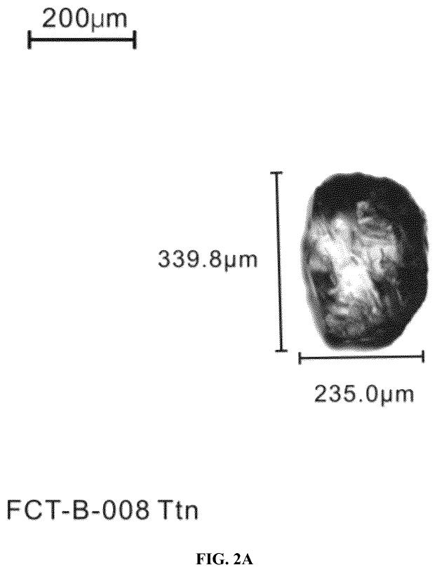

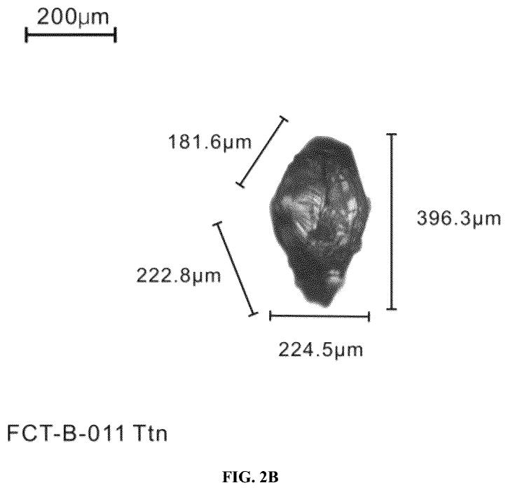

5 clean FCT titanite particles without cracks and inclusions were selected under a microscope, and photographed and measured (FIG. 2A and FIG. 2B). Each titanite particle was loaded into a niobium tube with a length and a diameter both being about 1 mm.

### (2) Helium Content Analysis

The extraction and analysis of ⁴He in each titanite sample was conducted on Alphachron helium isotope MS. Each titanite sample was heated in a 970 nm diode laser to extract ⁴He, and then a helium content was determined by QMG QMS.

5 titanite samples encapsuled in niobium tubes were placed in a laser chamber of the helium isotope MS. Because the loading of the samples made the laser chamber exposed to an atmosphere, the laser chamber needed to be restored to a vacuum state before the heating and extracting ⁴He were started. After being vacuumized by a mechanical pump for 3 min and then by a turbo-molecular pump for 18 h, a vacuum degree in the laser chamber was lower than 1.0×10⁻⁸ Torr meeting an experimental need, and an experiment was then started.

Before determining a content of ⁴He in a titanite sample, cold blank and hot blank tests were performed. The cold blank test referred to measuring a content of ⁴He in an instrument tube when no laser heating was conducted under the same experimental procedure; and the hot blank test referred to measuring a content of ⁴He in the instrument tube when an empty niobium tube was heated by a laser under the same experimental procedure. Results of multiple batches of blank tests show that ⁴He contents in the cold blank and hot blank tests were always maintained at about 0.0010 ncc to 0.0040 ncc (Table 1). However, contents of ⁴He in the titanite samples all were 100 to 1,000 times or more the ⁴He contents of the blanks, and thus the blanks had no impact on a measurement of ⁴He in a titanite sample.

TABLE 1
⁴He analysis results of cold blank and hot blank tests

| Blank | ⁴He/ncc |
|---|---|
| CB-1 | 0.0019 |
| Nb HB-1 | 0.0021 |
| CB-2 | 0.0012 |
| Nb HB-2 | 0.0017 |
| CB-3 | 0.0020 |
| Nb HB-3 | 0.0040 |
| CB-4 | 0.0013 |
| Nb HB-4 | 0.0015 |
| CB-5 | 0.0017 |
| Nb HB-5 | 0.0019 |

Notes:
In Table 1, CB-n (n is 1 to 5) represents a cold blank test, and Nb HB-n (n is 1 to 5) represents a hot blank test.

Each titanite sample was heated for 10 min with a laser current of 15 A to allow gas extraction. Gas extraction was conducted two times for each titanite sample. After the two times of gas extraction were completed, analysis and calculation were conducted. When an amount of a gas extracted at the second time was smaller than 1% of an amount of a gas extracted at the first time, the gas extraction was completed.

### (3) Sample Dissolution

1) The 5 titanite samples each were transferred to a 4.5 mL PTFE sample-dissolving bottle. A sample-dissolving bottle in which a titanite sample was placed was called a sample bottle. A blank bottle was also provided, and an empty niobium tube was placed in the blank bottle for a whole-process blank test. A standard solution bottle was also provided, in which 25 μL of a standard solution including 25×10⁻⁹ of ²³⁸U and 25×10⁻⁹ of ²³²Th was placed. A matrix of the standard solution was nitric acid having a volume concentration of 10%.

2) 25 μL of a blank solution was added to the blank bottle, the blank solution being a concentrated nitric acid having a volume concentration of 50%.

3) 25 μL of a spike was added to each of the sample bottles and the standard solution bottle, respectively. The spike included ²³⁵U and ²³⁰Th, and a matrix of the spike was a concentrated nitric acid having a volume concentration of 50%.

4) 350 μL of purified hydrofluoric acid was added to each of the sample bottles, the blank bottle, and the standard solution bottle, respectively.

5) The sample bottles, the blank bottle, and the standard solution bottle each were placed in an autoclave, and 420 μL of concentrated nitric acid and 9 mL of hydrofluoric acid were added to the autoclave.

6) The autoclave was sealed and heated in an oven at 180° C. for 24 h.

7) After heating, the autoclave has been cooled and then taken out from the oven, and the sample bottles, the blank bottle, and the standard solution bottle were taken out, placed on a heating plate, and heated at 60° C. to evaporate all liquid therein.

8) After all liquid in each bottle was evaporated, 300 μL of concentrated hydrochloric acid was added to each bottle. The bottles were placed in an autoclave, and 9 mL of concentrated hydrochloric acid was added to the autoclave. The autoclave was sealed again and heated in an oven at 180° C. for 24 h.

9) After the re-dissolution was completed, a solution in each bottle was transferred by a pipette to a 7 mL PTFE sample-dissolving bottle, and resulting 7 mL sample-dissolving bottles were placed on a heating plate and heated at 80° C. When a volume of a solution in each sample-dissolving bottle was reduced to 100 μL, the heating was stopped. The sample-dissolving bottles each were cooled, and then 300 μL of ultrapure water (UPW) was added to each sample-dissolving bottle for dilution to obtain solutions for uranium-thorium content analysis. The solutions each were transferred from a sample-dissolving bottle to a 1.5 mL centrifuge tube for MS analysis.

### (4) Uranium-Thorium Content Analysis

Uranium and thorium content analysis was conducted on ICP-MS. Counts of isotopes with mass numbers of 230, 232, 235, and 238 were mainly tested. A blank count was deducted from counts of isotopes in a sample to obtain 230/232 and 235/238 ratios, and ²³⁸U and ²³²Th contents were calculated based on a standard solution.

The determined contents of ⁴He, ²³⁸U, and ²³²Th were substituted into an age equation to calculate a (uranium-thorium)/helium age of FCT titanite. (Uranium-thorium)/helium age results of FCT titanite are shown in Table 2.

### Example 2

Example 2 was performed roughly the same as Example 1, except that the FCT titanite had a different particle size. (Uranium-thorium)/helium age results were shown in Table 2.

### Example 3

Example 3 was performed roughly the same as Example 2, except that the FCT titanite had a different particle size. (Uranium-thorium)/helium age results were shown in Table 2.

TABLE 2
Comparison of U-Th/He age results of FCT titanite
samples with reference values in the existing literature

| | | Effective | | |
|---|---|---|---|---|
| | | radius | | |
| Sample name | | (μm) | Th/U | Age (Ma) |
| Example 1 | 1 | 245 | 4.5-4.7 | 28.69 ± 0.90 |
| | 2 | 188 | | |
| | 3 | 212 | | |
| | 4 | 230 | | |
| | 5 | 175 | | |
| Example 2 | 6 | 192 | 4.14-4.76 | 28.87 ± 0.82 |
| | 7 | 238 | | |
| | 8 | 270 | | |
| | 9 | 195 | | |
| | 10 | 218 | | |
| Example 3 | 11 | 208 | | |
| | 12 | 248 | | |
| | 13 | 248 | 4.29-4.76 | 28.97 ± 0.85 |
| | 14 | 292 | | |
| | 15 | 195 | | |

| | | | |
|---|---|---|---|
| Reference value 1: | — | — | 30.1 ± 1.0 |
| test of FCT titanite | | | |
| through dissolution | | | |
| of aliquots | | | |
| Reference value 2: | — | 4.35-5.3 | 27.98 ± 0.86 |
| test of FCT titanite by | | | |
| a laser in-situ micro- | | | |
| analysis method | | | |

The results in Table 1 show that the method according to the present disclosure has excellent applicability to different titanite samples. Specifically, titanite particles of different particle sizes were adopted in Examples 1 to 3, and corresponding age results were consistent in an error range and were also consistent with the reference values in an error range, indicating that the (uranium-thorium)/helium dating method according to the present disclosure is reliable and an age measured by the method is repeatable. The dissolution method according to the present disclosure makes it possible to completely dissolve titanite particles of various particle sizes, including large-size titanite particles each with a radius of greater than 250 μm.

In Table 2, reference value 1 is derived from: Reiners P W, Farley K A, 1999. Helium diffusion and (U-Th)/He thermochronometry of titanite. Geochimica et Cosmochimica Acta, 63 (22): 3845-3859; and

reference value 2 is derived from: Alexandra M. Hornea, Matthijs C. van Soest, Kip V. Hodges, et al, 2016. Integrated single crystal laser ablation U/Pb and (U-Th)/He dating of detrital accessory minerals—Proof-of-concept studies of titanites and zircons from the Fish Canyon tuff. Geochimica et Cosmochimica Acta, 178:106-123.

In order to acquire the optimal heating temperature, heating time, and re-dissolution conditions for the sample dissolution process, a plurality of comparative examples were set. Comparative Examples 1 to 6 are control tests set for conditions of the initial digestion. Comparative Examples 7 and 8 are control tests set for conditions of re-dissolution.

### Comparative Example 1

1) The 5 titanite samples each were transferred to a 4.5 mL PTFE sample-dissolving bottle. A sample-dissolving bottle in which a titanite sample was placed was called a sample bottle. A blank bottle was also provided, and an empty niobium tube was placed in the blank bottle for a whole-process blank test. A standard solution bottle was also provided, in which 25 μL of a standard solution was placed.

2) 25 μL of a blank solution was added to the blank bottle.

3) 25 μL of a spike was added to each of the sample bottles and the standard solution bottle.

4) 350 μL of purified hydrofluoric acid was added to each of the sample bottles, the blank bottle, and the standard solution bottle.

5) The sample bottles, the blank bottle, and the standard solution bottle each were placed in an autoclave, and 420 μL of concentrated nitric acid and 9 mL of hydrofluoric acid were added to the autoclave.

6) The autoclave was sealed and heated in an oven at 220° C. for 60 h to allow digestion.

7) The autoclave was cooled and then taken out from the oven, and the sample bottles, the blank bottle, and the standard solution bottle were taken out, placed on a heating plate, and heated at 60° C. to evaporate all liquid therein.

8) After evaporating all liquid in each bottle, 300 μL of concentrated hydrochloric acid was added to each bottle. The bottles were placed in an autoclave, and 9 mL of concentrated hydrochloric acid was added to the autoclave. The autoclave was sealed and heated in an oven at 180° C. for 24 h to allow re-dissolution.

9) After the re-dissolution was completed, a solution in each bottle was transferred by a pipette to a 7 mL PTFE sample-dissolving bottle, and resulting 7 mL sample-dissolving bottles were placed on a heating plate and heated at 80° C. When a volume of a solution in each sample-dissolving bottle was reduced to 100 μL, the heating was stopped. The sample-dissolving bottles each were cooled, and then 300 μL of UPW was added to each sample-dissolving bottle for dilution to obtain solutions for uranium-thorium content analysis. The solutions each were transferred from a sample-dissolving bottle to a 1.5 mL centrifuge tube for MS analysis.

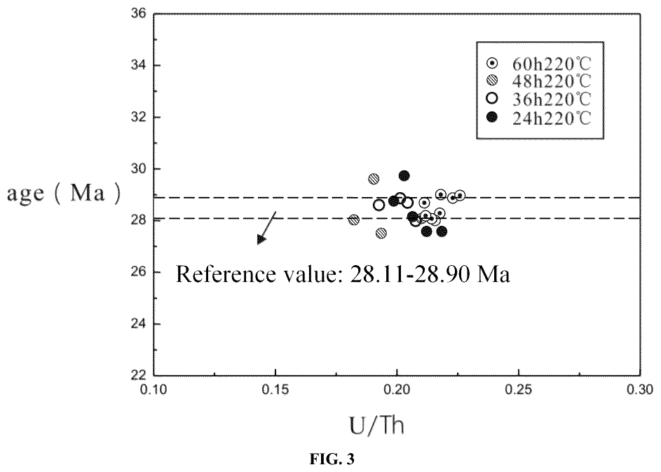

Final (uranium-thorium)/helium age results of FCT titanite are shown in FIG. 3.

### Comparative Example 2

Comparative Example 2 was performed roughly the same as Comparative Example 1, except that the initial digestion was conducted at 220° C. for 48 h. Final (uranium-thorium)/helium age results of FCT titanite are shown in FIG. 3.

### Comparative Example 3

Comparative Example 3 was performed roughly the same as Comparative Example 1, except that the initial digestion was conducted at 220° C. for 36 h. Final (uranium-thorium)/helium age results of FCT titanite are shown in FIG. 3.

### Comparative Example 4

Comparative Example 4 was performed roughly the same as Comparative Example 1, except that the initial digestion was conducted at 220° C. for 24 h. Final (uranium-thorium)/helium age results of FCT titanite are shown in FIG. 3.

### Comparative Example 5

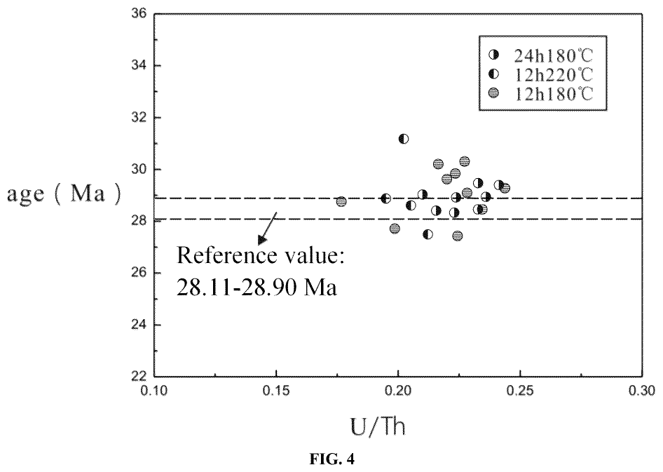

Comparative Example 5 was performed roughly the same as Comparative Example 1, except that the initial digestion was conducted at 220° C. for 12 h. Final (uranium-thorium)/helium age results of FCT titanite are shown in FIG. 4.

### Comparative Example 6

Comparative Example 6 was performed roughly the same as Comparative Example 1, except that the initial digestion was conducted at 180° C. for 12 h. Final (uranium-thorium)/helium age results of FCT titanite are shown in FIG. 4.

### Comparative Example 7

1) The 5 titanite samples each were transferred to a 4.5 mL PTFE sample-dissolving bottle. A sample-dissolving bottle in which a titanite sample was placed was called a sample bottle. A blank bottle was also provided, in which an empty niobium tube was placed for a whole-process blank test. A standard solution bottle was also provided, in which 25 μL of a standard solution was placed.

2) 25 μL of a blank solution was added to the blank bottle.

3) 25 μL of a spike was added to each of the sample bottles and the standard solution bottle.

4) 350 μL of purified hydrofluoric acid was added to each of the sample bottles, the blank bottle, and the standard solution bottle.

5) The sample bottles, the blank bottle, and the standard solution bottle each were placed in an autoclave, and 420 μL of concentrated nitric acid and 9 mL of hydrofluoric acid were added to the autoclave.

6) The autoclave was sealed and heated in an oven at 180° C. for 24 h.

7) The autoclave was cooled and then taken out from the oven, and the sample bottles, the blank bottle, and the standard solution bottle were taken out, placed on a heating plate, and heated at 60° C. to evaporate all liquid therein.

8) After evaporating all liquid in each bottle, 300 μL of concentrated hydrochloric acid was added to each bottle. The bottles were placed in an autoclave, and 9 mL of concentrated hydrochloric acid was added to the autoclave. The autoclave was sealed and heated in an oven at 220° C. for 24 h to allow re-dissolution.

9) After the re-dissolution was completed, a solution in each bottle was transferred by a pipette to a 7 mL PTFE sample-dissolving bottle, and resulting 7 mL sample-dissolving bottles were placed on a heating plate and heated at 80° C. When a volume of a solution in each sample-dissolving bottle was reduced to 100 μL, the heating was stopped. The sample-dissolving bottles each were cooled, and then 300 μL of UPW was added to each sample-dissolving bottle for dilution to obtain solutions for uranium-thorium content analysis. The solutions each were transferred from a sample-dissolving bottle to a 1.5 mL centrifuge tube for MS analysis.

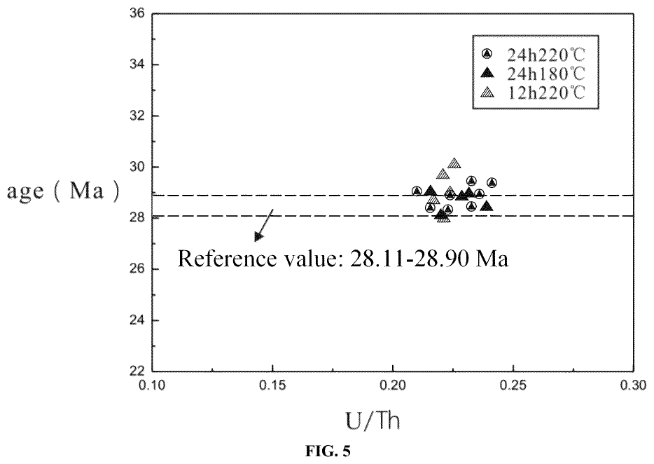

Final (uranium-thorium)/helium age results of FCT titanite are shown in FIG. 5.

### Comparative Example 8

Comparative Example 8 was performed roughly the same as Comparative Example 1, except that the re-dissolution was conducted at 220° C. for 12 h. Final (uranium-thorium)/helium age results of FCT titanite are shown in FIG. 5.

It can be seen from the control tests that, when the heating for the initial digestion is conducted for more than 24 h and the heating for the initial digestion is conducted at a temperature higher than 180° C., resulting (uranium-thorium)/helium ages of FCT titanite samples are consistent with the reference values within an error range, and a discrete degree is low. When a heating time for the initial digestion is 12 h, ages obtained at a heating temperature of either 180° C. or 220° C. for the initial digestion have a high discrete degree, and the ages tend to increase with the increase of a U/Th ratio, indicating that the titanite particles may not be completely dissolved, making uranium and thorium contents obtained through experimental analysis lower than actual uranium and thorium contents. When the heating time for the initial digestion is greater than 24 h and the heating temperature for the initial digestion is higher than 180° C., the titanite could be completely dissolved. However, considering the experimental efficiency, environmental protection, and energy conservation, the heating temperature of 180° C. and the heating time of 24 h are deemed as the optimal conditions for the initial digestion in the present disclosure.

For the re-dissolution, control tests are also set to obtain the optimal conditions. When heating for the re-dissolution is conducted at 220° C. for 12 h, resulting ages of the titanite samples are consistent with the reference values within an error range, but a discrete degree is high and the ages tend to slightly increase with the increase of a U/Th ratio. In order to guarantee an accuracy of an experimental result, the heating temperature of 220° C. and the heating time of 12 h are abandoned. Ages obtained by heating at 180° C. for 24 h are consistent with the reference values within an error range and a discrete degree is low. Therefore, the heating temperature of 180° C. and the heating time of 24 h are deemed as the optimal conditions for the re-dissolution in the present disclosure.

The above descriptions are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

1. A method for dissolving a single-particle titanite, comprising the steps of:
mixing the single-particle titanite, hydrofluoric acid, and a concentrated nitric acid to obtain a mixture, and subjecting the mixture to thermal digestion at 180° C. for 24 h in an autoclave, to obtain a primary dissolved sample, the concentrated nitric acid having a volume concentration of 50%;
heating the primary dissolved sample and evaporating all liquid therein, to obtain a dry sample; and
mixing the dry sample with a concentrated hydrochloric acid, and subjecting a resulting mixture to re-dissolution in an autoclave at 180° C. for 24 h, to obtain a re-dissolved sample.

2. The method as claimed in claim 1, wherein the hydrofluoric acid is added in an amount of 350 μL, and the concentrated nitric acid is added in an amount of 25 μL.

3. The method as claimed in claim 1, wherein the concentrated hydrochloric acid is added in an amount of 300 μL.

4. The method as claimed in claim 1, wherein the hydrofluoric acid, the concentrated nitric acid, and the concentrated hydrochloric acid each independently contain lower than 0.01 ppb of a metal impurity.

5. The method as claimed in claim 1, wherein heating the primary dissolved sample and evaporating all liquid therein is conducted at 60° C.

6. A method for determining an age of a single-particle titanite by (uranium-thorium)/helium dating, comprising the steps of
S1: selecting a single-particle titanite sample;
S2: heating the single-particle titanite sample and extracting ⁴He therefrom, and purifying a resulting gas to obtain a purified gas; and determining a content of ⁴He in the purified gas by an isotope dilution method using a helium isotope mass spectrometer (MS), namely a content of ⁴He in the single-particle titanite sample;
S3: dissolving the single-particle titanite sample according to the method as claimed in claim 1 to obtain a mixed solution to be tested, wherein the thermal digestion is conducted as follows: mixing the single-particle titanite sample with a spike and hydrofluoric acid to obtain a first mixture, and subjecting the first mixture to the thermal digestion; and determining contents of ²³⁸U and ²³²Th in the single-particle titanite sample by an isotope dilution method using an inductively coupled plasma mass spectrometer (ICP-MS), wherein the spike is a concentrated nitric acid solution comprising ²³⁵U, ²³⁸U, ²³²Th, and ²³⁰Th; and
S4: substituting determined contents of ⁴He, ²³⁸U, and ²³²Th in the single-particle titanite sample into age equation (1), and calculating a (uranium-thorium)/helium age of the single-particle titanite sample,
$$equation ( 1 )$$
$${}^{4} He = 8 \times {}^{238} U \times ( {e}^{{λ}_{238} t} - 1 ) + 7 \times ( {}^{238} U / 137.88 ) ( {e}^{{λ}_{235} t} - 1 ) + 6 \times {}^{232} Th \times ( {e}^{{λ}_{232} t} - 1 ) ,$$
wherein in equation (1), ⁴He, ²³⁸U, and ²³²Th each represent a measured number of atoms; t represents an accumulated time of a radioactive decay for producing a daughter isotope ⁴He; and λ₂₃₈, λ₂₃₅, and λ₂₃₂ represent decay constants of ²³⁸U, ²³⁵U, and ²³²Th, respectively, which are 1.55125×10⁻¹⁰ a⁻¹, 9.8485×10⁻¹⁰ a⁻¹, and 4.9475×10⁻¹¹ a⁻¹, respectively.

7. The method as claimed in claim 6, wherein the single-particle titanite sample has a minimum width of larger than 80 μm.

8. The method as claimed in claim 6, wherein heating the single-particle titanite sample and extracting ⁴He therefrom is conducted in a 970 nm diode laser with a laser current of 15 A for 10 min.

9. The method as claimed in claim 6, wherein in S2, determining the content of ⁴He in the purified gas comprises the steps of:
mixing the purified gas with a spike ³He to obtain a sample mixed gas, and determining a ⁴He/³He ratio in the sample mixed gas using a helium isotope MS, which is denoted as (⁴He/³He)_{Spiked Sample};
mixing a ⁴He standard gas in a known amount with the spike ³He to obtain a standard mixed gas, and determining a ⁴He/³He ratio in the standard mixed gas using the helium isotope MS, which is denoted as (⁴He/³He)_{Spike Q standard}, wherein a volume of the spike ³He used for preparation of the sample mixed gas is the same as a volume of the spike ³He used for preparation of the standard mixed gas; and
calculating a content of ⁴He in the purified gas according to equation (2):
$$equation ( 2 )$$
$${}_{}^{4} {He}_{Sample}^{} = {}_{}^{4} {He}_{Q Standard}^{} \times [ {( {}_{}^{4} He / {}_{}^{3} He )}_{Spiked Sample} / {( {}_{}^{4} He / {}_{}^{3} He )}_{Spike Q Standard} ] ,$$
wherein in equation (2), ⁴He_{Sample} represents a content of ⁴He in the purified gas; and
⁴He_{Q Standard} represents a content of ⁴He in the ⁴He standard gas.

10. The method as claimed in claim 7, wherein in S2, determining the content of ⁴He in the purified gas comprises the steps of:
mixing the purified gas with a spike ³He to obtain a sample mixed gas, and determining a ⁴He/³He ratio in the sample mixed gas using a helium isotope MS, which is denoted as (⁴He/³He)_{Spiked Sample};
mixing a ⁴He standard gas in a known amount with the spike ³He to obtain a standard mixed gas, and determining a ⁴He/³He ratio in the standard mixed gas using the helium isotope MS, which is denoted as (⁴He/³He)_{Spike Q standard}, wherein a volume of the spike ³He used for preparation of the sample mixed gas is the same as a volume of the spike ³He used for preparation of the standard mixed gas; and
calculating a content of ⁴He in the purified gas according to equation (2):
$$equation ( 2 )$$
$${}_{}^{4} {He}_{Sample}^{} = {}_{}^{4} {He}_{Q Standard}^{} \times [ {( {}_{}^{4} He / {}_{}^{3} He )}_{Spiked Sample} / {( {}_{}^{4} He / {}_{}^{3} He )}_{Spike Q Standard} ] ,$$
wherein in equation (2), ⁴He_{Sample} represents a content of ⁴He in the purified gas; and
⁴He_{Q Standard} represents a content of ⁴He in the ⁴He standard gas.

11. The method as claimed in claim 6, wherein S3 comprises:
providing the concentrated nitric acid solution comprising ²³⁵U, ²³⁸U, ²³²Th, and ²³⁰Th as the spike, wherein a ²³⁵U/²³⁸U ratio and a ²³⁰Th/²³²Th ratio in the spike are calibrated;
providing a nitric acid solution with known ²³⁸U and ²³²Th contents and no ²³⁰Th as a standard solution, wherein a ²³⁵U/²³⁸U ratio in the standard solution is calibrated;
mixing the single-particle titanite sample with the spike and the hydrofluoric acid to obtain the first mixture, and subjecting the first mixture to the thermal digestion, the heating and evaporating, and the re-dissolution with the concentrated hydrochloric acid sequentially to obtain the mixed solution to be tested;
mixing the standard solution with the spike and the hydrofluoric acid to obtain a third mixture, and subjecting the third mixture to the thermal digestion, the heating and evaporating, and the re-dissolution with the concentrated hydrochloric acid sequentially to obtain a spike/standard solution mixture, wherein a volume of the spike used for preparation of the mixed solution to be tested is the same as a volume of the spike used for preparation of the spike/standard solution mixture;
determining a ²³⁵U/²³⁸U ratio and a ²³⁰Th/²³²Th ratio in the mixed solution to be tested and the spike/standard solution mixture using the ICP-MS;
according to equation (3), calculating a content of ²³⁸U in the spike, which is denoted as ²³⁸U_{Spike}; and then according to equation (4), calculating a content of ²³⁸U in the single-particle titanite sample, which is denoted as ²³⁸U_{Sample}:
$${}_{}^{238} {U}_{Spike}^{} = {}_{}^{238} {U}_{Standard} \times \frac{{( \frac{{}^{235} U}{{}^{238} U} )}_{Standard} - {( \frac{{}^{235} U}{{}^{238} U} )}_{mix}}{{( \frac{{}^{235} U}{{}^{238} U} )}_{mix} - {( \frac{{}^{235} U}{{}^{238} U} )}_{Spike}} , \qquad equation ( 3 )$$
wherein in equation (3), ²³⁸U_{Standard} represents a definite number of ²³⁸U atoms added from the standard solution to the spike/standard solution mixture; (²³⁵U/²³⁸U)_{Standard} represents calibrated ²³⁵U/²³⁸U ratio in the standard solution; (²³⁵U/²³⁸U)_{Spike} represents calibrated ²³⁵U/²³⁸U ratio in the spike; and (²³⁵U/²³⁸U)ₘᵢₓ represents a ²³⁵U/²³⁸U ratio in the spike/standard solution mixture determined by the ICP-MS; and
$${}_{}^{238} {U}_{Sample}^{} = {}_{}^{238} {U}_{Spike} \times \frac{{( \frac{{}^{235} U}{{}^{238} U} )}_{spike - sample} - {( \frac{{}^{235} U}{{}^{238} U} )}_{Spike}}{{( \frac{{}^{235} U}{{}^{238} U} )}_{Sample} - {( \frac{{}^{235} U}{{}^{238} U} )}_{spike - sample}} , \qquad equation ( 4 )$$
wherein in equation (4), ²³⁸U_{Spike} represents a definite number of ²³⁸U atoms added from the spike to the spike/standard solution mixture, which is calculated according to equation (3), and equivalent to a definite number of ²³⁸U atoms added from the spike to the mixed solution to be tested; (²³⁵U/²³⁸U)_{Spike} represents calibrated ²³⁵U/²³⁸U ratio in the spike; (²³⁵U/²³⁸U)_{Sample} represents a natural ²³⁵U/²³⁸U ratio in the single-particle titanite sample; and (²³⁵U/²³⁸U)ₛₚᵢₖₑ₋ₛₐₘₚₗₑ represents a ²³⁵U/²³⁸U ratio in the mixed solution to be tested determined by the ICP-MS; and
according to equation (5), calculating a content of ²³²Th in the spike, which is denoted as ²³²Th_{Spike}; and then according to equation (6), calculating a content of ²³²Th in the single-particle titanite sample, which is denoted as ²³²Th_{Sample}:
$${}_{}^{232} {Th}_{Spike}^{} = {}_{}^{232} {Th}_{Standard} \times \frac{{( \frac{{}^{230} Th}{{}^{232} Th} )}_{mix}}{{( \frac{{}^{230} Th}{{}^{232} Th} )}_{Spike} - {( \frac{{}^{230} Th}{{}^{232} Th} )}_{mix}} , \qquad equation ( 5 )$$
wherein in equation (5), a ²³⁰Th/²³²Th ratio in the standard solution is 0; ²³²Th_{Standard} represents a definite number of ²³²Th atoms added from the standard solution to the spike/standard solution mixture; (²³⁰Th/²³²Th)_{Spike} represents calibrated ²³⁰Th/²³²Th ratio in the spike; and (²³⁰Th/²³²Th)ₘᵢₓ represents a ²³⁰Th/²³²Th ratio in the spike/standard solution mixture determined by the ICP-MS; and
$${}_{}^{232} {Th}_{Sample}^{} = {}_{}^{232} {Th}_{Spike} \times \frac{{( \frac{{}^{230} Th}{{}^{232} Th} )}_{Spike} - {( \frac{{}^{230} Th}{{}^{232} Th} )}_{spike - sample}}{{( \frac{{}^{230} Th}{{}^{232} Th} )}_{spike - sample}} , \qquad equation ( 6 )$$
wherein in equation (6), a ²³⁰Th/²³²Th ratio in the single-particle titanite sample is 0; ²³²Th_{Spike} represents a definite number of ²³²Th atoms added from the spike to the spike/standard solution mixture, which is calculated according to equation (5), and equivalent to a definite number of ²³²Th atoms added from the spike to the mixed solution to be tested; (200Th/²³²Th)_{Spike} represents calibrated ²³⁰Th/²³²Th ratio in the spike; and (²³⁰Th/²³²Th)ₛₚᵢₖₑ₋ₛₐₘₚₗₑ represents a ²³⁰Th/²³²Th ratio in the mixed solution to be tested determined by the ICP-MS.
